(19) United States
(12) Patent Application Publication
(10) Pub. No.: US 2014/0005527 A1
(43) Pub. Date: Jan. 2, 2014

(54) METHOD AND SYSTEM FOR DYNAMIC REFERENCING AND REGISTRATION USED WITH SURGICAL AND INTERVENTIONAL PROCEDURES

(71) Applicants: Kaustubh Ravindra Nagarkar, Clifton Park, NY (US); Daniel Eduardo Groszmann, Belmont, MA (US)

(72) Inventors: Kaustubh Ravindra Nagarkar, Clifton Park, NY (US); Daniel Eduardo Groszmann, Belmont, MA (US)

(73) Assignee: General Electric Company, Schenectady, NY (US)

(21) Appl. No.: 13/538,595

(22) Filed: Jun. 29, 2012

(51) Int. Cl. A61B 6/12

(52) U.S. Cl. 600/424

(57) ABSTRACT

A medical reference and registration system includes a substrate configured for placement on an external surface of a patient such that the substrate conforms to and moves with the external surface of the patient. The medical reference and registration system also includes multiple integrated sensor-fiducial marker devices mounted on the substrate. Each integrated sensor-fiducial marker device includes a sensor and at least one fiducial marker coupled to the sensor. Each sensor is pre-aligned with respect to the at least one fiducial marker so that a location of each fiducial marker is known with respect to its respective sensor.

### BACKGROUND

The subject matter disclosed herein relates generally to invasive or minimally invasive procedures, such as surgical or interventional procedures. In particular, the subject matter relates to image-guided computer-assisted procedures employing various imaging modalities and a navigation system.

As medical imaging technologies have matured, it has become possible to combine the use of medical imaging techniques with computer-assisted navigation technology to aid in the performance of invasive or minimally invasive procedures. For example, invasive or minimally invasive procedures, such as certain surgical or interventional procedures, may benefit from the use of computer-assisted imaging and navigation techniques that allow a clinician to visualize the internal or obscured structures in the surgical area while the procedure is being performed. In this way, the clinician may perform the desired surgical or interventional procedure with a greater chance of success and without unnecessary tissue damage.

In practice, such image-guided computer-assisted techniques typically employ a tracking frame or reference device placed proximate to the anatomy of interest. The reference device moves with the patient to provide accurate and consistent tracking of the anatomy of interest after a registration between the image and the patient is performed. For procedures involving bony anatomy, the reference device is typically secured rigidly to the bony anatomy of interest via an invasive or minimally invasive procedure (e.g., screwed on or in the patient). However, in soft tissue procedures using image-guided computer-assisted techniques, there is no bony anatomy for securely attaching the reference device. Lack of bony anatomy also may complicate the registration step necessary for computer-assisted navigation in these medical procedures. Lack of a combined patient reference and registration device typically complicates testing the accuracy of the navigation system during a trouble shooting process.

### BRIEF DESCRIPTION

In accordance with a first embodiment, a medical reference system includes a substrate configured for placement on an external surface of a patient such that the substrate conforms to and moves with the external surface. The medical reference system also includes multiple integrated sensor-fiducial marker devices mounted on the substrate. Each integrated sensor-fiducial marker device includes a sensor and at least one fiducial marker coupled to the sensor. Each sensor is pre-aligned with respect to the at least one fiducial marker so that a location of each fiducial marker is known with respect to its respective sensor.

In accordance with a second embodiment, a medical reference system includes a substrate configured for placement on an external surface of a patient such that the substrate conforms to and moves with the external surface. The medical reference system also includes multiple integrated sensor-fiducial marker devices mounted on the substrate. Each integrated sensor-fiducial marker device includes a magnetoresistance sensor and at least one fiducial marker coupled to the magnetoresistance sensor. The magnetoresistance sensor is configured to measure a position of the respective integrated sensor-fiducial marker device along x, y, and z coordinates.

In accordance with a third embodiment, a method for dynamic referencing and registration of patient images includes the following acts. The method includes generating one or more images or volumetric representations based on image data acquired using an imaging system. At least three fiducial markers of at least one integrated sensor-fiducial marker device are visible within the one or more images or volumetric representations. The at least one integrated sensor-fiducial marker includes a magnetoresistance sensor and the at least three fiducial markers coupled to the magnetoresistance sensor. The method also includes distinguishing the fiducial markers within the one or more images or volumetric representations based on an overall pattern of the fiducial markers or distinctive geometry of each fiducial marker. The method further includes generating a first set of position data for the magnetoresistance sensor of the at least one integrated sensor-fiducial marker device to determine a position of each of the at least three fiducial markers. The method yet further includes generating a second set of position data for the at least three fiducial markers based solely on their location in the one or more images or volumetric representations having the visible fiducial markers. The method still further includes registering the second set of position data of the at least three fiducial markers referencing the one or more images or volumetric representations with the first set of position data of the at least three fiducial markers.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a schematic view of an embodiment of an integrated sensor-fiducial marker device;

FIG. 2 is a schematic view of an embodiment of a dynamic reference device that includes multiple integrated sensor-fiducial marker devices of FIG. 1 mounted on a conformable substrate;

FIG. 3 is a schematic view of an embodiment of a dynamic reference device that includes multiple integrated sensor-fiducial marker devices of FIG. 1 mounted on multiple layers of a conformable substrate;

FIG. 4 is a schematic view of an embodiment of components of an imaging system and a navigation system;

FIG. 5 is a schematic view of an embodiment of components of a computed tomography or three-dimensional fluoroscopy imaging system and a navigation system; and

FIG. 6 is a flow chart of an embodiment of a method for dynamic referencing and registration of patient images with a navigation system to guide surgical and interventional devices.

### DETAILED DESCRIPTION

The disclosed embodiments are directed to dynamic referencing of internal anatomical structures or organs for image-guided procedures using a dynamic reference device placed on an external surface of a patient. In particular, the disclosed embodiments include a conformable member or substrate (e.g., rigid-flex substrate) that includes multiple integrated sensor-fiducial marker devices mounted on the substrate. Each integrated sensor-fiducial marker device includes a sensor (e.g., magnetoresistance sensor) and at least one fiducial marker coupled to the sensor. The at least one fiducial marker is pre-aligned with the sensor so that a location of each fiducial marker is known with respect to its respective sensor. This enables patient registration without having to reference each fiducial marker with its respective sensor. There are at least three fiducial markers to register the patient to the images. The sensor enables the measurement of a position (x, y, and z coordinates) and orientation (roll, pitch, and yaw) and of its respective integrated sensor-fiducial marker device. Thus, each sensor operates with six degrees of freedom. The dynamic reference device enables automatic registration of a patient with their image data using a navigation system to guide surgical or interventional devices. In addition, the reference device enables non-invasive dynamic referencing since it is disposed on the external surface of the patient. For a rigid substrate, only a single magnetoresistance sensor is needed since all the fiducial markers are rigid with respect to the sensor. For a flexible substrate, multiple sensors are needed as the fiducial markers integrated into one sensor can move with respect to the fiducial markers integrated into a second sensor.

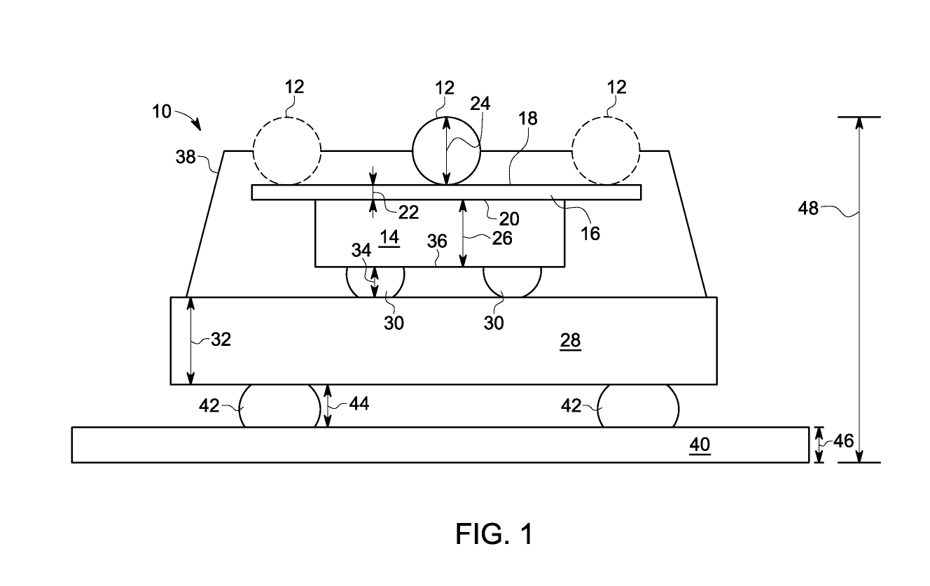

For example, referring to FIG. 1, an integrated sensor-fiducial marker device 10 is depicted that is suitable for use with a dynamic reference device. As depicted, the integrated sensor-fiducial marker device 10 is arranged in a ball grid array package. In certain embodiments, the integrated sensor-fiducial marker device 10 may be arranged in a chip scale package, or a wafer level package chip scale package. In yet other embodiments, the integrated sensor-fiducial marker device 10 may be arranged in a lead frame package, or a no-lead package such as Quad Flat No-lead (QFN) package. The integrated sensor-fiducial marker device 10 includes one or more imageable pattern markers or fiducial markers 12 coupled to a magnetoresistance sensor 14. In certain embodiments, the fiducial markers 12 may be radio-opaque markers. The one or more fiducial markers 12 are pre-aligned with the magnetoresistance sensor 14 so that a location of each fiducial marker 12 is known with respect to its respective magnetoresistance sensor 14. This enables patient registration without having to reference each fiducial marker 12 with its respective magnetoresistance sensor 14. As depicted, the one or more fiducial markers 12 are coupled to the magnetoresistance sensor 14 via a substrate 16. The substrate 16 may be organic, plastic, or ceramic. In particular, the one or more fiducial markers 12 are coupled to a first surface 18 of the substrate 16, while the magnetoresistance sensor 14 is coupled to the second surface 20 (e.g., opposite surface) of the substrate 16. In certain embodiments, patterned metallization may be performed on the surface 18 of the substrate 16. For example, depending on the material of the substrate 16 used, an adhesive layer (e.g., titanium or titanium oxide), solderable layer (e.g., copper), a diffusion barrier layer (e.g., electroless nickel), and/or a corrosion resistance layer (e.g., gold) may be added via the patterned metallization. Alternatively, the surface 18 may include coppers pads with gold for the coupling of the markers 12. The magnetoresistance sensor 14 may be coupled to the substrate 16 via a standard epoxy. In certain embodiments, the magnetoresistance sensor 14 may be coupled to the substrate 16 via a solder process. A thickness 22 of the substrate 16 may range from approximately 25 μm to 1 mm. In certain embodiments, the one or more fiducial markers 12 may be directly coupled to the magnetoresistance sensor 14 (e.g., without substrate 16). In some embodiments, a prefabricated (e.g., at the wafer level) substrate with the one or more fiducial markers 12 may be attached to the magnetoresistance sensor 14.

The fiducial markers 12 for each integrated sensor-fiducial marker device 10 may be arranged in a distinct pattern (e.g., nonsymmetrical pattern) to distinguish the fiducial markers 12 on one device 10 from fiducial markers 12 on the other devices 10. In addition or alternatively, the one or more fiducial markers 12 for each device 10 may include a distinctive geometry to distinguish between the fiducial markers 12 on the devices 10. As depicted, the fiducial markers 12 include a spherical shape. In certain embodiments, the fiducial markers 12 may be a metal BB or solder bump. A diameter 24 of the fiducial markers 12 may range from approximately 1 to 2 mm. In other embodiments, the fiducial markers 12 may include other shapes (e.g., cube, pyramid, etc.).

In certain embodiments, the sensor element 14 for each integrated sensor-fiducial marker device 10 includes a magnetoresistance sensor. In such an embodiment, the magnetoresistance sensor 14 provides a change in electrical resistance of a conductor or semiconductor when a magnetic field is applied. The magnetoresistance sensor 14 enables the position measurement of its respective integrated sensor-fiducial marker device 10 along axes in an x, y, and z direction. In addition, the magnetoresistance sensor 14 enables the orientation measurement of a roll, pitch, and yaw. Thus, the magnetoresistance sensor 14 operates with six degrees of freedom. In certain embodiments, the magnetoresistance sensor 14 utilizes anisotropic magnetoresistive based-technology. A thickness 26 of the magnetoresistance sensor 14 may range from approximately 50 to 200 μm. In some embodiments, the thickness 26 of the magnetoresistance sensor may also range from approximately 50 to 750 μm.

In certain embodiments, the magnetoresistance sensor 14 may include an area array or peripheral array interconnect flip chip magnetoresistance sensor 14. The flip chip structure of the magnetoresistance sensor 14 is coupled to a rigid substrate 28 (e.g. printed circuit board (PCB)) as well as connectors to external circuitry via reflow solder balls 30. The rigid substrate 28 may include a thickness 32 of approximately 1 mm, while the solder balls 30 may include a thickness 34 of approximately 50 μm when flowed. In certain embodiments, under bump metallization may be performed on a surface 36 of the sensor 14. For example, depending on the material of the die used as the base of the sensor 14, an adhesive layer (e.g., titanium oxide), solderable layer (e.g., copper), a diffusion layer (e.g., electroless nickel), and/or a corrosion resistance layer (e.g., gold) may be added via the under bump metallization. The fiducial markers 12, sensor element 14, and/or substrate 16 are encapsulated on the rigid substrate 28 via an injection molded encapsulation 38. Thermoplastics, thermosets, and/or elastomers may be used in the encapsulation. In other embodiments, the magnetoresistance sensor 14 may include an area array or peripheral array interconnect Chip Scale Package (CSP), or Wafer-level Chip Scale Package (WLCSP).

The rigid substrate 28 is coupled to another substrate 40 via reflow solder ball joints 42. The substrate 40 may be a rigid or flexible substrate (e.g., PCB). The reflow solder ball joints may include a thickness 44 of approximately 0.3 mm when flowed. The substrate 40 may include a thickness 46 of approximately 0.8 to 1.5 mm. Overall, the integrated sensor-fiducial marker device 10 includes a thickness 48 ranging from approximately 4 to 9 mm.

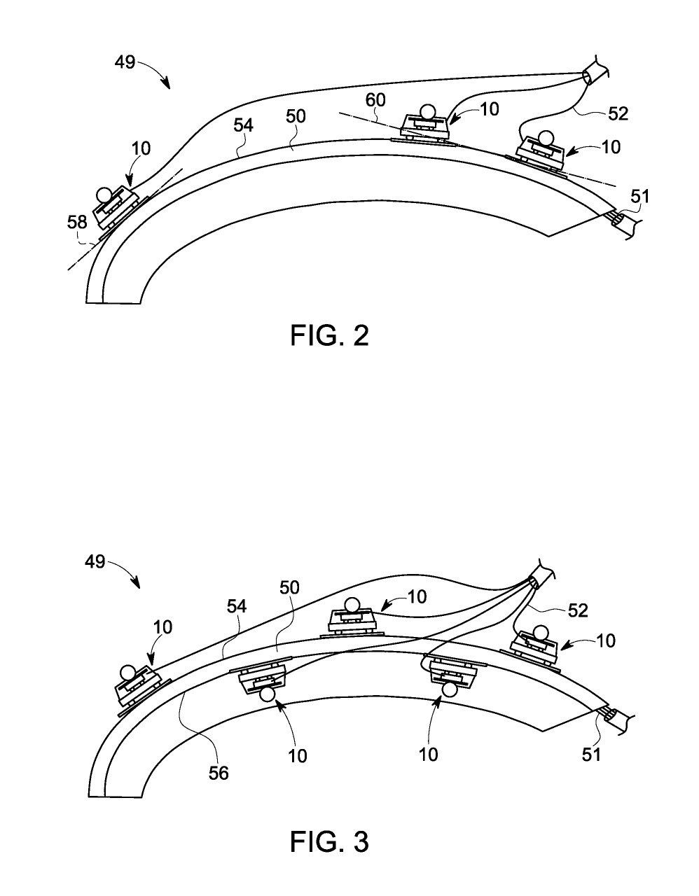

As illustrated in FIG. 2, a dynamic reference device or surgical/interventional reference system 49 includes a plurality of the integrated sensor-fiducial marker devices 10 mounted on a single layer 54 of a rigid-flex substrate 50. In certain embodiments, the rigid-flex substrate 50 may include devices mounted on multiple layers (see FIG. 3). As illustrated, at least three devices 10 are mounted on the substrate 50. In certain embodiments, more than three devices 10 may be mounted on the substrate 50. Each mounted integrated sensor-fiducial marker device 10 is self-aligned on the substrate 50 via reflow soldered ball joints. As depicted, the integrated sensor-fiducial marker devices 10 are mounted in a nonsymmetrical pattern on the substrate 50. The nonsymmetrical arrangement of the devices 10 helps identify the orientation of the dynamic reference device 49 via the fiducial markers 12 visible in the acquired image data (e.g., images or volumetric representations). In addition, in certain embodiments, the devices 10 are arranged in a non-planar arrangement (e.g., disposed on more than one plane such as planes 58, 60) to improve the accuracy away from any one plane 58, 60.

The rigid-flex substrate 50 is also designed to conform to and move with the external surface. This enables the use of the dynamic reference device 49 in non-invasive dynamic referencing since it is disposed on the external surface of the patient. The rigid-flex substrate 50 may be made of laminate layers that include organic layers, fiberglass weave layers, copper layers (which may be converted into traces), and/or a gold layer covering. The devices 10 may be mounted on to pads disposed on the substrate 50 via reflow soldering. Wiring 51 is disposed in the rigid-flex substrate 50 to facilitate the transmission of signals to the devices 10 mounted on the dynamic reference device 49 from components of a navigation system (e.g., power circuitry, control circuitry, etc.). This enables the devices 10 to receive power and input signals such as calibration signals. In addition, as depicted, each sensor element 14 of each device 10 is coupled to conductive wiring 52 from components of the navigation system (e.g., power circuitry, control circuitry, etc.). This enables the devices 10 to transmit output signals (e.g., representative of position and orientation data for the sensor element) to these components.

As mentioned above, the integrated sensor-fiducial marker devices 10 may be disposed on more than one layer of the rigid-flex substrate 50. FIG. 3 depicts an embodiment of the dynamic reference device 49 that includes multiple devices 10 disposed on multiple layers of the substrate 50. In general, the dynamic reference device 49 is as described in FIG. 2. The device 49 illustrated in FIG. 3 includes three integrated sensor-fiducial marker devices 10 mounted on a first layer 54 of the substrate 50 and two devices 10 mounted on a second layer 56 of the substrate 50. In certain embodiments, any particular layer 54, 56 of the substrate 50 may include one or more devices 10. In total, the dynamic reference device 49 includes at least one integrated sensor-fiducial device 10 mounted on the multiple layers of the substrate 50 and at least three fiducial markers on the overall dynamic reference device 49.

As described above, the dynamic reference device 49 may be used in accordance with the present technique to allow image-guided computer-assisted invasive or minimally invasive procedures. As will be appreciated, any imaging modality suitable for use in an image-guided procedure may be employed in the present technique. Examples of such imaging modalities include X-ray based imaging techniques which utilize the differential attenuation of X-rays to generate images (such as three-dimensional fluoroscopy, computed tomography (CT), tomosynthesis techniques, and other X-ray based imaging technologies). Other exemplary imaging modalities suitable for image-guided procedures may include magnetic resonance imaging (MRI), ultrasound or thermoacoustic imaging techniques, and/or optical imaging techniques. Likewise, nuclear medicine imaging techniques (such as positron emission tomography (PET) or single positron emission computed tomography (SPECT)) that utilize radiopharmaceuticals may also be suitable imaging technologies for performing image-guided procedures. Likewise, combined imaging modality systems, such as PET/CT systems, may also be suitable for performing image-guided procedures as described herein. Therefore, throughout the present discussion, it should be borne in mind that the present techniques are generally independent of the system or modality used to acquire the image data. That is, the technique may operate on stored raw, processed or partially processed image data from any suitable source.

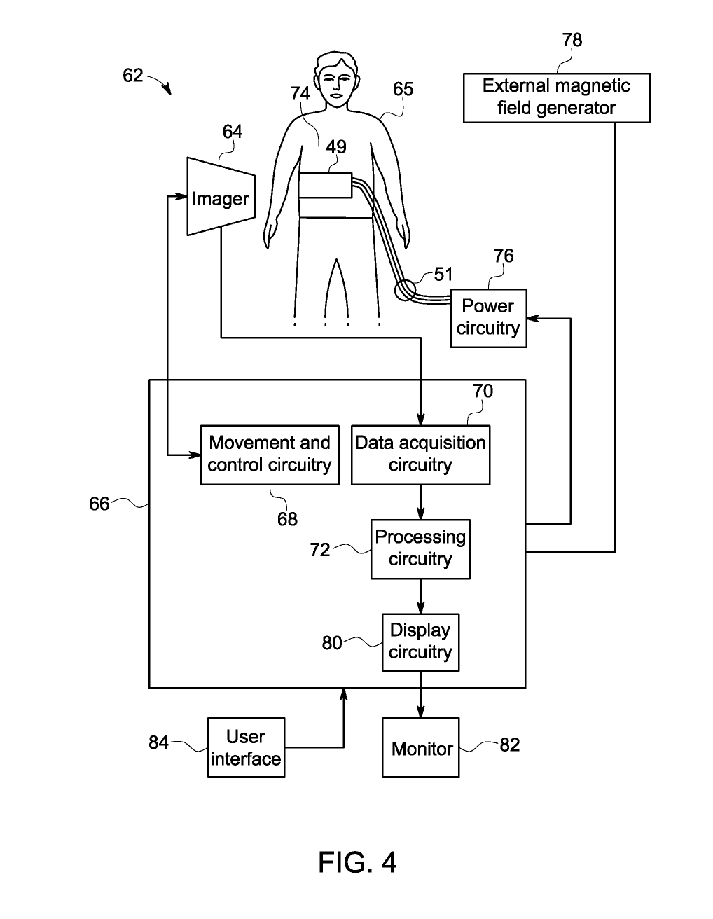

For example, turning now to FIG. 4, an overview of an exemplary generalized imaging system 62, which may be representative of various imaging modalities, is depicted. The generalized imaging system 62 typically includes some type of imager 64 which detects signals and converts the signals to useful data. As described more fully below, the imager 64 may operate in accordance with various physical principles for creating the image data. In general, however, image data indicative of regions of interest in a patient 65 are created by the imager 64 in a digital medium for use in image-guided procedures.

The imager 64 may be operated by system control circuitry 66 which controls various aspects of the imager operation and acquisition and processing of the image data as well as dynamic reference registration data acquired using the present techniques. In the depicted generalized embodiment, the system control circuitry 66 includes movement and control circuitry 68 useful in operating the imager 64. For example, the movement and control circuitry 68 may include radiation source control circuits, timing circuits, circuits for coordinating the relative motion of the imager 64 (such as with regard to a patient support and/or detector assembly), and so forth. The imager 64, following acquisition of the image data or signals, may process the signals, such as for conversion to digital values, and forwards the image data to data acquisition circuitry 70. For digital systems, the data acquisition circuitry 70 may perform a wide range of initial processing functions, such as adjustment of digital dynamic ranges, smoothing or sharpening of data, as well as compiling of data streams and files, where desired. The data are then transferred to data processing circuitry 72 where additional processing and analysis are performed. For the various digital imaging systems available, the data processing circuitry 72 may perform substantial reconstruction and/or analyses of data, ordering of data, sharpening, smoothing, feature recognition, and so forth.

In addition to processing the image data, the processing circuitry 72 may also receive and process motion or location information (e.g., received via the wires 51) related to an anatomical region of interest. In the depicted embodiment, the dynamic reference device 49 is placed on an external surface 74 of the patient 38. The dynamic reference device 49, as discussed above, is provided with numerous (for example, one or more) integrated sensor-fiducial marker devices 10 with magnetoresistance sensors 14 (see FIGS. 1-3) configured to provide position and orientation information. In certain embodiments where the sensor elements 14 are powered, the magnetoresistance sensors 14 may be connected, such as via one or more conductive wires 51, to suitable power circuitry 76, such as an electrical power source or outlet or a suitable battery. While in the depicted embodiment the power circuitry 76 is depicted as being separate from the system control circuitry 66, in other embodiments the power circuitry 76 may be part of the system control circuitry 66.

In the depicted embodiment, an alternating current (AC) or pulsed direct current (DC) magnetic field is generated by an external magnetic field generator 78 (e.g., electromagnetic transmitter). The magnetoresistance sensors 14 (e.g., magnetoresistance sensor) of the integrated sensor-fiducial marker devices 10 generate signals representative of location data (including position and/or orientation data) in response to the applied magnetic field, which are sent via the wires 51 to the system control circuitry 66 (in particular the processing circuitry 72). As discussed in greater detail below, the location data may be used in conjunction with the image data to facilitate an image-guided procedure.

The processed image data and/or location data may be forwarded to display circuitry 80 for display at a monitor 82 for viewing and analysis. While operations may be performed on the image data prior to viewing, the monitor 82 is at some point useful for viewing reconstructed images derived from the image data collected. The images may also be stored in short or long-term storage devices which may be local to the imaging system 62, such as within the system control circuitry 66, or remote from the imaging system 62, such as in picture archiving communication systems. The image data can also be transferred to remote locations, such as via a network.

For simplicity, certain of the circuitry discussed above, such as the movement and control circuitry 68, the data acquisition circuitry 70, the processing circuitry 72, and the display circuitry 80, are depicted and discussed as being part of the system control circuitry 66. Such a depiction and discussion is for the purpose of illustration only, however, and is intended to merely exemplify one possible arrangement of this circuitry in a manner that is readily understandable. Those skilled in the art will readily appreciate that in other embodiments the depicted circuitry may be provided in different arrangements and/or locations. For example, certain circuits may be provided in different processor-based systems or workstations or as integral to different structures, such as imaging workstations, system control panels, and so forth, which functionally communicate to accomplish the techniques described herein.

The operation of the imaging system 62 may be controlled by an operator via a user interface 84 which may include various user input device, such as a mouse, keyboard, touch screen, and so forth. Such a user interface may be configured to provide inputs and commands to the system control circuitry 66, as depicted. Moreover, it should also be noted that more than a single user interface 84 may be provided. Accordingly, an imaging scanner or station may include an interface which permits regulation of the parameters involved in the image data acquisition procedure, whereas a different user interface may be provided for manipulating, enhancing, and viewing resulting reconstructed images.

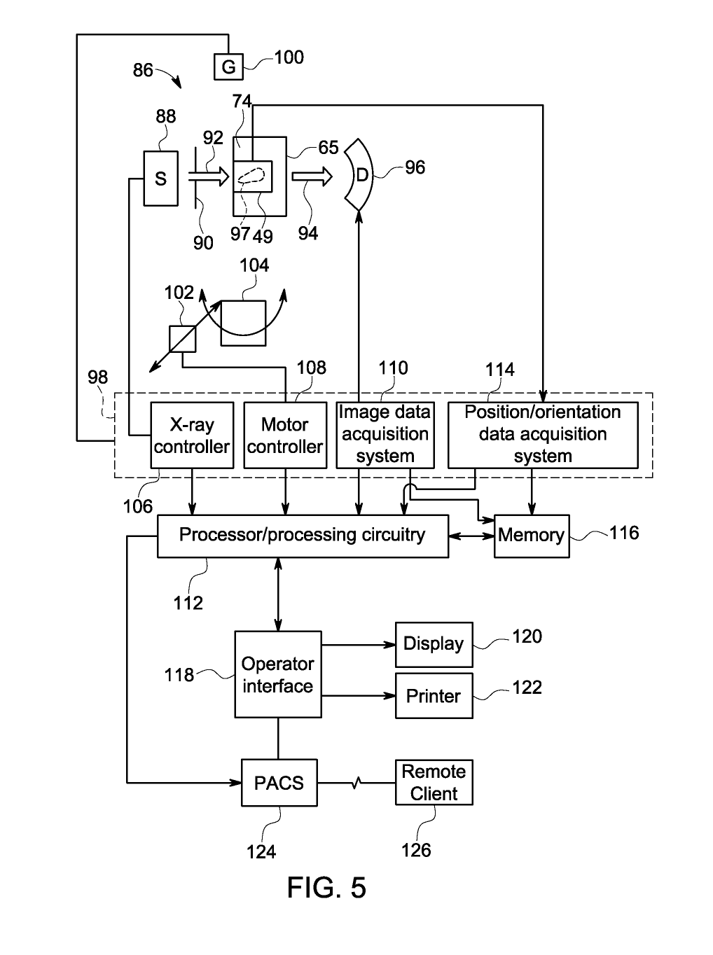

To discuss the technique in greater detail, a specific medical imaging modality based generally upon the overall system architecture outlined in FIG. 4 is depicted in FIG. 5, which generally represents an X-ray based system 86. It should be noted that, while reference is made in FIG. 5 to an X-ray based system, the present technique also encompasses other imaging modalities, as discussed above, such as MRI, PET, SPECT, ultrasound, and so forth.

In the depicted exemplary embodiment, FIG. 5 illustrates diagrammatically an X-ray based imaging system 86 for acquiring and processing image data. In the illustrated embodiment, imaging system 86 is a computed tomography (CT) system or three-dimensional fluoroscopy imaging system designed to acquire X-ray projection data, to reconstruct the projection data into a two or three-dimensional image, and to process the image for display and analysis in accordance with the present technique. In the embodiment illustrated in FIG. 5, X-ray based imaging system 86 includes a source of X-ray radiation 88 positioned adjacent to a collimator 90. The X-ray source 88 may be a standard X-ray tube or one or more solid-sate X-ray emitters.

In the depicted embodiment, the collimator 90 permits a stream of radiation 92 to pass into a region in which a subject, such as the patient 38 is positioned. The stream of radiation 92 may be generally fan or cone shaped, depending on the configuration of the detector array as well as the desired method of data acquisition. A portion of the radiation 94 passes through or around the patient 38 and impacts a detector array, represented generally as reference numeral 96. Detector elements of the array produce electrical signals that represent the intensity of the incident X-ray beam. The signals generated by the detector array 96 may be subsequently processed to reconstruct a visual representation (i.e., an image or volumetric representation) of the features within the patient 38. For example, images of the anatomical structure of interest 97 may be reconstructed in the depicted embodiment.

A variety of configurations of the detector 96 may be employed in conjunction with the techniques described herein. For example, the detector 96 may be a multi-row detector, such as a detector having eight or sixteen rows of detector elements, which achieves limited longitudinal coverage of the object or patient being scanned. Similarly, the detector 96 may be an area detector, such as a high-resolution radiographic detector having hundreds of rows of detector elements, which allows positioning of the entire object being imaged within the field of view of the system 86. Regardless of the configuration, the detector 96 enables acquisition and/or measurement of the data used in image reconstruction of the anatomical structure of interest.

The source 88 is controlled by a system controller 98, which furnishes both power, and control signals for examination procedures. Moreover, the detector 96 is coupled to the system controller 98, which commands acquisition of the signals generated in the detector 86. The system controller 98 may also execute various signal processing and filtration functions, such as for initial adjustment of dynamic ranges, interleaving of digital image data, and so forth. In general, system controller 98 commands operation of the imaging system 86 to execute examination protocols and to process acquired data. In the present context, system controller 98 also includes signal processing circuitry, typically based upon a general purpose or application-specific digital computer, associated memory circuitry for storing programs and routines executed by the computer (such as programs and routines for implementing the present technique), as well as configuration parameters and image data, interface circuits, and so forth. The system controller 98 may also control an external magnetic field generator 100 (e.g., electromagnetic transmitter).

In the embodiment illustrated in FIG. 5, the system controller 98 is coupled to a linear positioning subsystem 102 and rotational subsystem 104. The rotational subsystem 104 enables the X-ray source 88, collimator 90 and the detector 86 to be rotated one or multiple turns around the patient 65. It should be noted that the rotational subsystem 104 might include a gantry or C-arm apparatus. Thus, the system controller 98 may be utilized to operate the gantry or C-arm. The linear positioning subsystem 102 typically enables a patient support, such as a table, upon which the patient rests, to be displaced linearly. Thus, the patient table may be linearly moved relative to the gantry or C-arm to generate images of particular areas of the patient 65.

Additionally, as will be appreciated by those skilled in the art, the source 88 of radiation may be controlled by an X-ray controller 106 disposed within the system controller 98. Particularly, the X-ray controller 106 may be configured to provide power and timing signals to the X-ray source 88. A motor controller 108 may also be part of the system controller 98 and may be utilized to control the movement of the rotational subsystem 104 and the linear positioning subsystem 102.

Further, the system controller 98 is also illustrated as including an image data acquisition system 110. In this exemplary embodiment, the detector 86 is coupled to the system controller 98, and more particularly to the image data acquisition system 110. The image data acquisition system 110 receives data collected by readout electronics of the detector 86. The image data acquisition system 110 typically receives sampled analog signals from the detector 96 and converts the data to digital signals for subsequent processing by processing circuitry 112, which may, for example, be one or more processors of a general or application specific computer.

As depicted, the system controller 98 also includes a position/orientation data acquisition system 114 configured to acquire position and orientation data from the dynamic reference device 49. In the depicted embodiment, the device 49 provides signals generated by magnetoresistance sensors 14 of the devices 10 (in response to an applied external magnetic field) on the substrate 50 placed on the external surface 74 of the patient 65 undergoing imaging. The position/orientation data acquisition system 114 processes signals acquired from the device 49 to generate position and/or orientation information about the magnetoresistance sensors 14 and/or fiducial markers 12 of the devices 10. The position and/or orientation information generated by the position/orientation data acquisition system 114 may be provided to the processing circuitry 112 and/or a memory 116 for subsequent processing.

The processing circuitry 112 is typically coupled to the system controller 98. The data collected by the image data acquisition system 110 and/or by the position/orientation data acquisition system 114 may be transmitted to the processing circuitry 112 for subsequent processing and visual reconstruction. The processing circuitry 112 may include (or may communicate with) the memory 116 that can store data processed by the processing circuitry 112 or data to be processed by the processing circuitry 112. It should be understood that any type of computer accessible memory device capable of storing the desired amount of data and/or code may be utilized by such an exemplary system 86. Moreover, the memory 116 may include one or more memory devices, such as magnetic or optical devices, of similar or different types, which may be local and/or remote to the system 86. The memory 114 may store data, processing parameters, and/or computer programs having one or more routines for performing the processes described herein.

The processing circuitry 112 may be adapted to control features enabled by the system controller 98, i.e., scanning operations and data acquisition. For example, the processing circuitry 112 may be configured to receive commands and scanning parameters from an operator via an operator interface 118 typically equipped with a keyboard and other input devices (not shown). An operator may thereby control the system 86 via the input devices. A display 120 coupled to the operator interface 118 may be utilized to observe a reconstructed visual representation. Additionally, the reconstructed image may also be printed by a printer 122, which may be coupled to the operator interface 118. As will be appreciated, one or more operator interfaces 118 may be linked to the system 86 for outputting system parameters, requesting examinations, viewing images, and so forth. In general, displays, printers, workstations, and similar devices supplied within the system may be local to the data acquisition components, or may be remote from these components, such as elsewhere within an institution or hospital, or in an entirely different location, linked to the image acquisition system via one or more configurable networks, such as the Internet, virtual private networks, and so forth.

The processing circuitry 112 may also be coupled to a picture archiving and communications system (PACS) 124. Image data generated or processed by the processing circuitry 96 may be transmitted to and stored at the PACS 124 for subsequent processing or review. It should be noted that PACS 124 might be coupled to a remote client 126, radiology department information system (RIS), hospital information system (HIS) or to an internal or external network, so that others at different locations may gain access to the image data.

The systems and devices described above may be utilized, as described herein, to provide dynamic referencing and registration for a region of interest of a patient undergoing an invasive or minimally invasive procedure, wherein the dynamic referencing registration device 49 is placed externally on the patient 65. In an exemplary embodiment, dynamically acquired position and orientation data for the region of the patient 65 (and magnetoresistance sensors 14 and/or fiducial markers 12) may be acquired using the non-invasive dynamic reference device 49 and the data may be automatically registered with concurrently or previously acquired image data without having to reference the fiducial markers 12 of the devices 10. In this way, the surgical/interventional procedure may be performed or guided based upon the dynamically referenced visual representation.

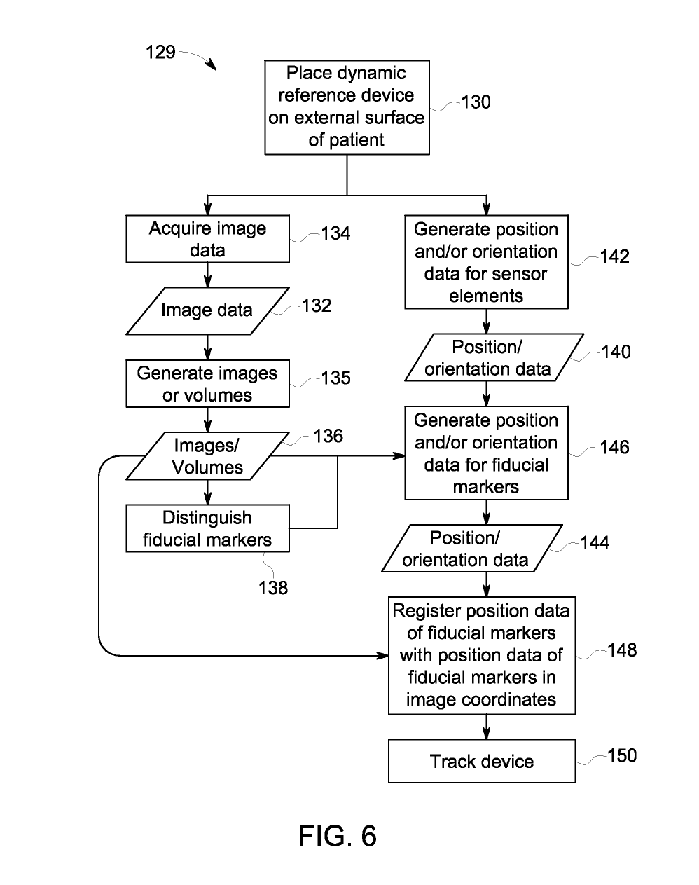

FIG. 6 illustrates a method 130 for dynamic referencing and automatic registration of patient images with a navigation system to guide surgical and interventional devices. The method 130 includes placing the conformable dynamic reference device 49 on an external surface of the patient 65 (block 130). Prior to an invasive or minimally invasive procedure, the image data 132 is acquired (block 134). As noted above, the image data 132 may be acquired using one or more suitable imaging modalities, such as three-dimensional fluoroscopy, CT, MRI, PET, X-ray, ultrasound and so forth. In the depicted embodiment, the image data 132 is used to generate (block 135) one or more visual representations, such as images or volumes 136 of a region of interest.

The fiducial markers 12 of the integrated sensor-fiducial marker devices 10 are visible within the images or volumes 136 of the region of interest. The method 130 includes distinguishing the fiducial markers 12 within the images or volumes 134 based on an overall pattern of the fiducial markers 12 of the devices 10 and/or a distinctive geometry of each fiducial marker 12 (block 138). Prior to, during, or after acquiring the image data 132 (block 134) and after placing the dynamic reference device 49 on the patient 65 (block 130), position and orientation data 140 is generated (e.g., in a reference frame of the navigation system) for the magnetoresistance sensors 14 of the devices 10 (block 142). Based solely on the position and orientation data 140 and the images or volumetric 136 with the visible fiducial markers 12, the position and/or orientation data 144 for the markers 12 is generated (block 146) due to the pre-alignment of the magnetoresistance sensors 14 and the markers 12 in the devices 10. Thus, the devices 10 provide automatic registration without having to reference each fiducial marker 12 with its respective magnetoresistance sensor 14. The method 130 includes registering the position and orientation data 144 for the markers 12 with the images or volumes 136 (block 148). In other words, the position and orientation data 144 for the markers 12 in navigation system coordinates is registered with the position and orientation data 140 in image coordinates.

An image-guided invasive or minimally invasive procedure may be performed using the registered images or volumes 136 and fiducial markers 14. In particular, once the previously acquired image-based information is registered to the measured position and/or orientation data of the dynamic reference device, changes in the position and/or orientation of the patient can be used to visually indicate changes to an image-based model. In other words, a displayed image of the anatomical region of interest may be updated, modified, altered, or otherwise, changed, based on the most current position and/or orientation data obtained from the magnetoresistance sensors 14 placed on external surface of the patient 65. In this way, even though no imaging processes are occurring during the operation, the previously acquired image data can be updated and manipulated to provide an accurate and current representation of the anatomical region undergoing the procedure.

Based on this registration between the images or volumes 136 (derived from the image data) and position and/or orientation data 144 for the fiducial markers (derived from the magnetoresistance sensor data), a surgical or interventional device may be tracked (Block 150) during an invasive or minimally invasive procedure, such as a surgical open or laparoscopic procedure. Examples of such surgical or interventional devices that may be tracked include instruments, implants, probes, awls, drills, aspirators, forceps, blades, screws, nails, pins, k-wires, needles, cannulas, introducers, catheters, guidewires, stents, heart valves, filters, endoscopes, laparoscopes, electrodes, and so forth. Typically the surgical or interventional device being tracked also includes a sensor, such as an electromagnetic sensor or a magnetoresistance sensor, so that position and orientation information for the surgical or interventional device is also acquired, thereby allowing the position of the surgical or interventional device to be displayed in conjunction with the registered image of the anatomical region of interest. In this manner, a system such as those described herein, may display to a user in real-time or substantially real-time the position and orientation of the device relative to the anatomical region of interest.

Technical effects of the disclosed embodiments include providing the dynamic reference device 49 that enables non-invasive dynamic referencing and patient registration. In particular, the device 49 is placed on the external surface 74 of the patient 65. In addition, the dynamic reference device 49 includes a plurality of integrated sensor-fiducial marker devices 10. Each integrated sensor-fiducial marker device 10 includes at least one fiducial marker 12 and the magnetoresistance sensor 14, which are pre-aligned with respect to each other, eliminating the need to reference the marker 12 and magnetoresistance sensor 14 with each other. Eliminating the reference step reduces manufacturing cost of the dynamic referencing registration system. In addition, workflow may be improved since the markers 12 are physically accessible to test the accuracy of the navigation system during a troubleshooting process.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A medical reference system comprising:
a substrate configured for placement on an external surface of a patient such that the substrate conforms to and moves with the external surface of the patient; and
a plurality of integrated sensor-fiducial marker devices mounted on the substrate, wherein each integrated sensor-fiducial marker device comprises:
a sensor; and
at least one fiducial marker coupled to the sensor, wherein the at least one fiducial marker is coupled to the sensor via an additional substrate with the sensor disposed on a first surface of the additional substrate and the at least one fiducial marker disposed on a second surface of the additional substrate opposite the first surface, and wherein each sensor is pre-aligned with respect to the at least one fiducial marker so that a location of each fiducial marker is known with respect to its respective sensor.

2. The medical reference system of claim 1, wherein each mounted integrated sensor-fiducial marker device is self-aligned on the substrate via reflow solder balls.

3. The medical reference system of claim 1, wherein the plurality of integrated sensor-fiducial marker devices is configured to provide automatic registration of patient images without having to reference each fiducial marker with its respective sensor.

4. The medical reference system of claim 1, wherein the sensor comprises a magnetoresistance sensor.

5. The medical reference system of claim 4, wherein the magnetoresistance sensor is arranged in a flip chip package.

6. The medical reference system of claim 4, wherein the magnetoresistance sensor is configured to measure a position of the respective integrated sensor-fiducial marker device along x, y, and z coordinates.

7. The medical reference system of claim 6, wherein the magnetoresistance sensor is configured to operate with six degrees of freedom.

8. The medical reference system of claim 1, comprising at least one integrated sensor-fiducial marker device mounted on the substrate and at least three fiducial markers in total.

9. The medical reference system of claim 1, wherein the additional substrate comprises an organic, ceramic, or plastic substrate.

10. (canceled)

11. The medical reference system of claim 1, wherein the plurality of integrated sensor-fiducial marker devices are mounted in a nonsymmetrical pattern on the substrate.

12. The medical reference system of claim 1, wherein each integrated sensor-fiducial marker device is arranged in a ball grid array package.

13. The medical reference system of claim 1, wherein the substrate comprises multiple layers, and the plurality of integrated sensor-fiducial marker devices are mounted on more than one layer of the substrate.

14. A medical reference system comprising:
a substrate configured for placement on an external surface of a patient such that the substrate conforms to and moves with the external surface of the patient; and
a plurality of integrated sensor-fiducial marker devices mounted on the substrate, wherein each integrated sensor-fiducial marker device comprises:
a magnetoresistance sensor; and
at least one fiducial marker coupled to the magnetoresistance sensor, wherein the at least one fiducial marker is coupled to the magnetoresistance sensor via an additional substrate with the magnetoresistance sensor disposed on a first surface of the additional substrate and the at least one fiducial marker disposed on a second surface of the additional substrate opposite the first surface, and wherein the magnetoresistance sensor is configured to measure a position of the respective integrated sensor-fiducial marker device along x, y, and z coordinates.

15. The medical reference system of claim 14, wherein the magnetoresistance sensor is pre-aligned with respect to the at least one fiducial marker so that a location of each fiducial marker is known with respect to its respective magnetoresistance sensor.

16. The medical reference system of claim 15, wherein each mounted integrated sensor-fiducial marker device is self-aligned on the substrate via reflow solder balls.

17. The medical reference system of claim 14, wherein each integrated sensor-fiducial marker device is configured to provide automatic registration of patient images without having to reference each fiducial marker with its respective magnetoresistance sensor.

18. The medical reference system of claim 14, wherein the magnetoresistance sensor is configured to operate with six degrees of freedom.

19. The medical reference system of claim 14, wherein the magnetoresistance sensor is arranged in a flip chip package.

20. The medical reference system of claim 14, comprising at least one integrated sensor-fiducial marker device mounted on the substrate and at least three fiducial markers in total.

21. The medical reference system of claim 14, wherein the plurality of integrated sensor-fiducial marker devices are mounted in a nonsymmetrical pattern on the substrate.

22. The medical reference system of claim 14, wherein each integrated sensor-fiducial marker device is arranged in a ball grid array package.

23. The medical reference system of claim 14, wherein the substrate comprises multiple layers, and the plurality of integrated sensor-fiducial marker devices are mounted on more than one layer of the substrate.

24. A method for dynamic referencing and registration of patient images comprising the acts of:
generating one or more images or volumetric representations based on image data acquired using an imaging system, wherein at least three fiducial markers of at least one integrated sensor-fiducial marker device are visible within the one or more images or volumetric representations, and the at least one integrated sensor-fiducial marker comprises a magnetoresistance sensor and the at least three fiducial markers coupled to the magnetoresistance sensor, and wherein the at least three fiducial markers are coupled to the magnetoresistance sensor via an additional substrate with the magnetoresistance sensor disposed on a first surface of the additional substrate and the at least three fiducial markers disposed on a second surface of the additional substrate opposite the first surface;
distinguishing the fiducial markers within the one or more images or volumetric representations based on an overall pattern of the at least three fiducial markers or distinctive geometry of each fiducial marker;
generating a first set of position data for the magnetoresistance sensor of the at least one integrated sensor-fiducial marker device to determine a position of each of the at least three fiducial markers;
generating a second set of position data for the at least three fiducial markers based solely on their location in the one or more images or volumetric representations having the visible fiducial markers; and
registering the second set of position data of the at least three fiducial markers referencing the one or more images or volumetric representations with the first set of position data of the at least three fiducial markers.

25. The method of claim 24, comprising tracking a surgical or interventional instrument, implant or device using the dynamically referenced and registered one or more images or volumetric representations.
